(19) United States
(12) Patent Application Publication
(10) Pub. No.: US 2018/0014949 A1
(43) Pub. Date: Jan. 18, 2018

(54) PROSTHETIC FOOT

(71) Applicants: KRAMSKI GmbH, Pforzheim (DE); Fraunhofer-Gesellschaft zur Förderung der angewandten Forschung e.V., München (DE)

(72) Inventors: Stefan Grosskopf, Straubenhardt (DE); Felix Starker, Stuttgart (DE); Andreas Kramski, Pforzheim (DE); Andreas Adler, Kraichtal (DE); Urs Schneider, Stuttgart (DE); Jannis Breuninger, Großbettlingen (DE)

(21) Appl. No.: 15/549,310

(22) PCT Filed: Feb. 4, 2016
(86) PCT No.: PCT/EP2016/052410

§ 371 Date: Aug. 7, 2017

(30) Foreign Application Priority Data
Feb. 6, 2015 (DE) 10 2015 101 746.6

(51) Int. Cl. A61F 2/66

(52) U.S. Cl. CPC A61F 2/66; A61F 2002/6671; A61F 2002/6614; A61F 2002/6642

(57) ABSTRACT

The invention relates to a prosthetic foot with a housing (12) on which a connector (15) for a below-knee prosthesis can be arranged, with a forefoot (21) and a heel (43), wherein the housing (12) and the forefoot (21) are connected by at least one forefoot spring element (28), wherein the forefoot (21) and the heel (43) are connected by at least one sole spring element (41), and wherein the heel (43) and the housing (12) are connected by at least one heel spring element (30), wherein the end portions belonging to the at least one forefoot spring element (28) and to the at least one sole spring element (41), and assigned to the forefoot (21), are positioned relative to each other with at least one holding mechanism (34) in such a way that the at least one forefoot spring element (28) and the at least one sole spring element (41) are guided movably relative to each other along their longitudinal axis during a rollover movement.

The invention relates to a prosthetic foot having a housing, on which a connector for a below-knee prosthesis can be arranged, and having a forefoot and a heel.

Document US 2004/0153168 A1 discloses a prosthetic foot which has a flexible sole with resilient elements mounted thereon so as to attain what is known as a roll-over shape, which is intended to give the user the feel of walking naturally. Such an embodiment of the prosthetic foot has the disadvantage that it is complex to produce. In addition, such a prosthetic foot is unsuitable for robust use. Furthermore, the adaptation of such a prosthetic foot to a specific user is complex.

Document WO 2014/147070 A discloses a prosthetic foot which has a forefoot spring element, a heel spring element, and a sole spring element. The forefoot spring element and the heel spring element are firmly screwed to each other in the region of a connector to the below-knee prosthesis. The further connections between the heel spring element and sole spring element and between the sole spring element and the forefoot spring element are provided by an adhesive bonding.

Document US 2012/0271434 A1 also discloses a prosthetic foot which is constructed in a number of parts. A sole element comprises a pocket in the region of the heel and a pocket in the region of the toes. Once the two-part heel spring element has been screwed in place, an end portion of the heel spring element is inserted into the pocket of the heel. A front end of the forefoot spring element is then fitted into the front pocket of the sole spring element, wherein each of the end portions of the forefoot spring element and heel spring element assigned to one another are then screwed together such that the forefoot spring element and the spring element are securely clamped in the pockets of the sole spring element.

The object of the invention is to create a prosthetic foot and a method for production thereof, which prosthetic foot enables economical production, is of simple design, and is robust and enables a simple adaptation of the mechanics to a specific user.

This object is achieved by a prosthetic foot in which a housing and a forefoot are connected by at least one forefoot spring element, the forefoot and a heel are connected by at least one sole spring element, and the heel and the housing are connected by at least one heel spring element, wherein the end portions of the at least one forefoot spring element and of the at least one sole spring element which are assigned to the forefoot are positioned in relation to one another, by means of at least one holding device, in such a way that the at least one forefoot spring element and the at least one sole spring element are guided displaceably relative to one another along their longitudinal axis during a movement of the prosthetic foot from heel to toe (roll-over movement). A rollover behaviour that comes very close to natural walking is thus made possible.

Furthermore, with the embodiment of the prosthetic foot according to the invention, the roll-over behaviour can be determined by the spring characteristic curve of the at least one forefoot spring element. A harmonic rolling between the heel and the forefoot can also be set primarily by the at least one sole spring element. A damped walking on the ground is determined substantially by the at least one heel spring element. Due to the use of spring elements of this type for the formation of the prosthetic foot, individual adaptation to a specific user can thus be made possible in a simple way. In addition, such spring elements can be produced economically and are also robust in use.

Furthermore, the end portions of the at least one forefoot spring element and of the at least one sole element which are assigned to the heel are preferably fixed firmly to one another by means of at least one holding device. When walking, defined ratios of the forces introduced are thus provided in the region of the heel, whereas harmonic and dynamic walking is attained with an increasing roll of the prosthetic foot from heel to toe by arranging the end portions of the at least one forefoot spring element and of the at least one sole element in the forefoot in such a way that these are slightly displaceable relative to one another.

Furthermore, the holding device of the forefoot and the holding device of the heel preferably have at least one slit-like or U-shaped holding portion for receiving the respective end portions of the at least one forefoot spring element and of the at least one sole spring element, and the end portions of the at least one sole spring element and of the at least one heel spring element. The receiving region for the end portions of the spring elements can thus be constructed almost identically. In addition, a releasable fastening can thus be created in a simple way.

The end portions of the at least one forefoot spring element and the at least one sole spring element which are assigned to the forefoot advantageously have a slot-like cutout or a U-shaped cutout, by which these end portions are fastened at least to the holding device of the forefoot by the preferably releasable connection element. In the region of the forefoot, the end portions of the spring elements assigned to one another are thus slightly displaceable relative to one another, whereby the roll-over behaviour of the prosthetic foot is improved.

Furthermore, the holding device of the forefoot preferably comprises at least one holding portion and one clamping element, which are preferably releasable relative to one another and form a receiving space for the end portions of the at least one forefoot spring element and of the at least one sole spring element. The holding portion and the clamping portion can preferably be arranged at a predetermined distance from each other such that the end portions of the at least one forefoot spring element and the at least one sole spring element are arranged therein without being pressed relative to one another. A sleeve is preferably clamped or held in a pressed manner between the holding portion and the clamping element such that this sleeve extends through the slot-like cut out or U-shaped cutout of the end portions of the at least one forefoot spring element or sole spring element and thus positions and/or fixes the end portions in relation to the sleeve or the receiving space in the forefoot so as to be displaceable relative to one another during a walking movement.

Preferably, the end portions of the at least one forefoot spring element and sole spring element are preferably releasably fastened in the forefoot, the end portions of the at least one sole spring element and heel spring element are preferably releasably fastened in the heel and the end portions of the at least one heel spring element and forefoot spring element are preferably releasably fastened to the housing. An almost modular construction can thus be provided for a prosthetic foot of this type, by means of which the spring elements arranged between the housing, the forefoot and the heel can be inserted, selected and/or exchanged in accordance with the number and/or design.

The at least one forefoot spring element, the at least one sole spring element, and the at least one heel spring element advantageously form a triangular structure or position the forefoot, the heel, and the housing in a triangular arrangement, wherein a substantially isosceles triangle is formed by the at least one forefoot spring element and the at least one sole spring element. As a result, not only is it possible to attain a compact structure and an optimised interaction between the spring elements in relation to the forces that are to be taken up, but in particular a structural design that is empathetic towards the body is created.

The at least one heel spring element preferably has a U-shaped or V-shaped extent, wherein the region of curvature of the U- or V-shaped extent points in the direction of the forefoot. As a result of this arrangement, a high take-up of forces as the heel touches down when the foot rolls from heel to toe can be made possible.

The spring elements are preferably produced from a strip-like spring element. The spring elements are preferably formed as leaf spring elements or are formed from a polymer composite element. In particular in the embodiment as leaf spring element, they can have a spring material thickness by way of example of from 0.1 mm to 3 mm. The spring material thickness and/or the number of layers of the spring elements can be adapted to the body weight and/or to the sporting activities of the individual wearing the prosthetic foot.

An imprint is preferably provided on the at least one forefoot spring element, between a portion of the at least one forefoot spring element lying firmly against the housing and the forefoot. By means of an imprint of this type, such as a bead or dome, a forefoot spring characteristic curve can be adjusted. In particular, a degressive spring behaviour can thus be set, whereby, when the foot rolls from heel to toe, just before push-off, in particular via a toe region, an increased springback effect is produced, which enables an additional energy recovery for the user. As a result of such an imprint, a snap-action effect, so to speak, of the at least one forefoot spring element can be attained, which leads to a delayed snapback in the standing phase of the walking cycle and thus allows dynamic walking. At the same time, by way of example, due to a degressive characteristic curve of the at least one forefoot spring element, the behaviour when walking is improved, since a more stable standing is thus provided on account of the initial gradient of the spring characteristic curve. In addition or alternatively, besides the imprint, the number of the forefoot spring elements, sole spring elements, and heel elements can also be selected and adapted accordingly, wherein, in addition and/or alternatively, the material selection and also the spring constants of the spring elements in addition and/or alternatively can be adapted and selected accordingly.

The housing of the prosthetic foot is formed in accordance with an advantageous embodiment of the invention as a foot upper part which has a connector portion for a connector to a below-knee prosthesis, from which a foot upper side extends in a wedge-like manner in the direction of the forefoot, with a bearing face provided on the housing on the sole side, which bearing face extends from the distal end of the foot upper side to the heel region and preferably runs in a rising manner to the heel. This housing, on the one hand, thus forms a secure and firm connection to a joint of the below-knee prosthesis and, on the other hand, enables a secure and defined bearing face of the at least one forefoot spring element and heel spring element to be provided. At the same time, the roll-over behaviour can be influenced by the shape of the foot upper part or bearing face thereof, since the at least one forefoot spring element is supported on this bearing face at least temporarily as the foot rolls from heel to toe.

The bearing face is advantageously curved, in particular in the distal region. Here, different radii can be provided, by means of which a limitation of the movement and also the extent of the bending of the at least one forefoot spring element can be set.

The housing preferably also has, distally on the bearing face, an end stop, which limits a curvature or bending of the forefoot spring element during a rollover movement. The forefoot spring element can thus be protected against overload.

Furthermore, a dome directed towards the forefoot spring element is preferably provided on the bearing face of the housing. This dome advantageously bears against the forefoot spring element in a starting position of the prosthetic foot. The forefoot spring element, in this region, can preferably also have an imprint, which in turn protrudes in the direction of the bearing face of the housing. The spring force can be increased as a result of this dome.

End portions of the at least one forefoot spring element and of the at least one heel element are preferably fastened in the heel region of the housing. The longest possible bearing face is thus provided at the housing so as to form a support for the at least one forefoot spring element and also so as to enable a harmonic rolling of the prosthetic foot from head to toe, which can be influenced by the profile or the extent of the bearing face. In particular, the bearing face is provided with a roll-over radius in the distal region.

A damping element is advantageously provided between the heel and the heel region of the housing and is preferably fastened to the housing by means of the releasable connection element, which fixes the end portions of the at least one heel spring element and forefoot spring element. By means of the combination of the at least one heel spring element and the damping element, the vibrations occurring with spring elements can be damped, such that a harmonic and dynamic walking is provided by a heel damping system of this type.

The damping element is preferably fixed by clamping between the heel region of the housing and the heel. This enables a design of simple structure, an exchange as necessary during the adaptation, and/or simplified maintenance.

A further advantageous embodiment of the prosthetic foot provides one or more heel spring elements arranged between the heel and the heel region of the housing, which heel spring elements can be modified between a rear position assigned to the heel and a front position shifted in the direction of the forefoot. The spring rigidity in the heel region can thus be modified. By way of example, it is lower if the heel spring elements are shifted in the direction of the forefoot.

Furthermore, a plurality of heel spring elements arranged between the heel and the heel region of the housing can be provided, which heel spring elements are arranged lying closely against one another or partially or completely fanned out in relation to one another. In the latter case, this means that each heel spring element is arranged at a distance from the adjacent heel spring element. As a result of this arrangement, the spring rigidity can also be modified. In particular in conjunction with the positioning of these heel spring elements closer to the heel region or closer to the forefoot, a very individual adaptation of the prosthetic foot to the individual wearing it can be made possible.

The at least one forefoot spring element extends freely, starting from a firm bearing against the bearing face in the heel region, to the forefoot, wherein, in particular during a roll-over movement of the foot from heel to toe, the at least one forefoot spring comes into full bearing against the bearing face. In addition to the at least one forefoot spring element, the roll-over behaviour can thus also be determined by the extent of the bearing face of the housing.

Between a firmly bearing portion of the at least one forefoot spring element and a distal end of the bearing face, a damping element forming the bearing face is provided. By way of example, this damping element can be formed as a damping cushion. Alternatively, a damping element which can be adjusted by the user can also be provided in order to set different hardnesses of the damping. By way of example, this can be an adjustable damping element. This damping element can also be formed as a layer formed from one or more elastomers, which for example can be glued, clamped, or injection moulded to the bearing face of the housing. By way of example, elastomer layers or soft-elastomer layers or materials can be provided. As a result of the interaction of this damping element with the at least one forefoot spring element, a forefoot damping system is achieved for reducing the vibrations for harmonic and dynamic walking. A noise reduction can also be attained.

A further advantageous embodiment of the invention provides that the at least one forefoot spring element comprises the imprint, in particular a bead or dome, which is oriented in the direction of the damping element, at least in a region opposite the damping element. This also enables a determination of the spring characteristic curve for the roll-over behaviour of the prosthetic foot.

At least one sole spring element advantageously extends beyond the forefoot and forms a toe region. The toe region is thus formed to be integrated with a sole spring element. In addition, as a result of this embodiment, the roll-over movement can be extended as far as the toe region.

Proceeding from the front end of the toe region, in particular from an upper side of the toe region, a strap, which is fixed at the front region of the toe region and in the heel region, extends preferably around the toe region and along an underside of the sole spring element as far as the heel, and from there as far as the heel region of the housing. This strap can be clamped at one end on one side and can be fixed releasably at the other end. This strap acts in the heel region as an Achilles tendon. In addition, a dynamic function can be attained as a result, in particular an additional energy recovery.

The strap, which extends from the front end of the toe region, along the sole spring element and the heel as far as the housing, is preferably connected at least at one end to a pre-tensioning device, by means of which a pre-tension acting on the damping element can be applied between a clamping point at the housing and at the toe region. By way of example, the strap can be fixedly clamped at the housing or at the toe region, in particular by clamping, adhesive bonding, riveting, or the like. The pre-tensioning device can be provided at the other end, which device can be easily actuated and tensions the strap between the clamping point and the pre-tensioning device. A pre-tension can thus be applied to the damping element in the heel region so that, for example, a damping is provided only from a predetermined load greater than the pre-tension of the damping element. A fine adjustment is thus possible with regard to the walking in the heel region, whereby the wearing comfort is increased.

The at least one forefoot spring element, sole element and/or heel element is formed from a high-grade steel, an aluminium alloy, a steel alloy, composite material, in particular iron-plastic composites, or from a soft metal, in particular copper, which preferably has a coating. Forged leaf springs can also be used as spring elements. The appropriate materials can be selected depending on the places of use and/or the weight of the user. The same is true for a coating. By way of example, a permanent coating, such as a Teflon coating, can be provided, whereby materials that are also more vulnerable to moisture or salts can be used for the spring element. Alternatively, removable coatings, such as greases, waxes, anti-corrosion materials or the like, can also be provided so as to protect the spring element against ambient influences. Furthermore, what are known as sandwich materials can also be used for the at least one spring element, for example a carbon composite, fibre-reinforced plastics, or the like.

The housing and/or the forefoot and/or the heel preferably consist of a light metal or a plastic or a composite material. In the case of a plastic material, unfilled or filled thermoplastic or thermosets or the like is preferably provided. Fibre-reinforced plastics can also be used. The housing and/or the forefoot and/or the heel are preferably produced as an injection-moulded part in order to keep the production costs low.

An advantageous structural embodiment provides that the forefoot, transversely to the longitudinal direction of the sole, is wider than the housing and/or the heel. As a result, the standing strength can be improved, and an increased assurance as the prosthetic foot rolls from heel to toe can also be attained.

The forefoot preferably has resiliently flexible supporting portions on one or both sides outside the receptacle for the end portions of the at least one forefoot spring element and the sole spring element. A lateral stabilisation or a transverse stabilisation of the prosthetic foot is thus made possible.

Furthermore, it is preferably provided that the housing has a ribbed portion comprising a plurality of ribs, at least on the foot upper side, wherein the rigidity of the housing can be adjusted by the extent and/or size and/or number of the ribs. A further parameter can thus be provided in order to enable an individual adaptation to the individual wearing the foot orthosis.

The object underlying the invention is furthermore achieved by a method for producing a prosthetic foot, in which method at least one forefoot spring element and heel spring element are connected to a housing, at least one heel spring element and sole spring element are connected to each other, and at least one forefoot spring element and sole spring element are connected to each other, wherein the at least one forefoot and heel spring element, the at least one forefoot and sole spring element, and the at least one sole and heel spring element are connected to each other by means of a releasable connection element. This has the advantage that a simple adaptation and design of the prosthetic foot to a specific user is made possible. In addition, a simple exchange of individual elements of the foot prosthesis can be made possible at the time of repair and/or maintenance.

In accordance with an advantageous embodiment of the method, the at least one forefoot, sole, and heel spring element are encased by a protective film, in particular a shrink film, and are then encapsulated in foam. This enables the prosthetic foot to be embodied in the form of a natural foot. In addition, a slight damping can be possible as a result of the selection of the foam material, polymer or vulcanisation to be applied.

A preferred embodiment of the method provides that a film is firstly placed into a shoe of the individual wearing the prosthetic foot, then the prosthetic foot is inserted, and the shoe is filled with foam, preferably integral foam. By introducing the film into the shoe before this is filled with foam, the prosthetic foot can be easily removed with the encapsulating foam from the shoe, such that the prosthetic foot is well adapted to this shoe. In addition, such foam-encapsulated prostheses also enable an improved hold in further shoes. Before the prosthetic foot is encapsulated in foam, a protective film is advantageously applied at least to the spring elements. When using an integral foam, the introduction of a release agent into the shoe instead of the lining with a film can be sufficient, since the integral foam has closed pores and forms an outer skin. The foam therefore does not enter into a connection to the inner side of the shoe and, once cured, can be easily removed from the shoe together with the foam-encapsulated prosthetic foot.

A further advantageous embodiment of the method provides that the film for lining the shoe is printed with a design corresponding to the external appearance of a foot. Once the shoe has been filled with foam, the inserted film can thus adhere to the foam and at the same time can mimic and visually present the natural form of the foot.

The invention and further advantageous embodiments and developments thereof will be described and explained in greater detail hereinafter by means of the examples depicted in the drawings. The features to be inferred from the description and the drawings can be applied individually or together in any combination according to the invention. In the drawings:

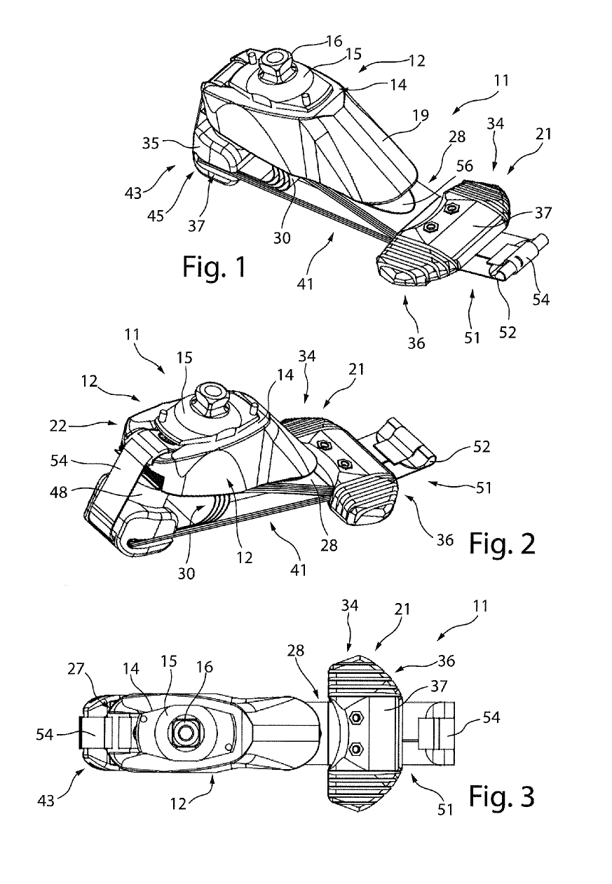

FIG. 1 shows a perspective view from the front and from above of a prosthetic foot,

FIG. 2 shows a perspective view from the rear and from above of the prosthetic foot according to FIG. 1,

FIG. 3 shows a view from above of the prosthetic foot according to Figure 1,

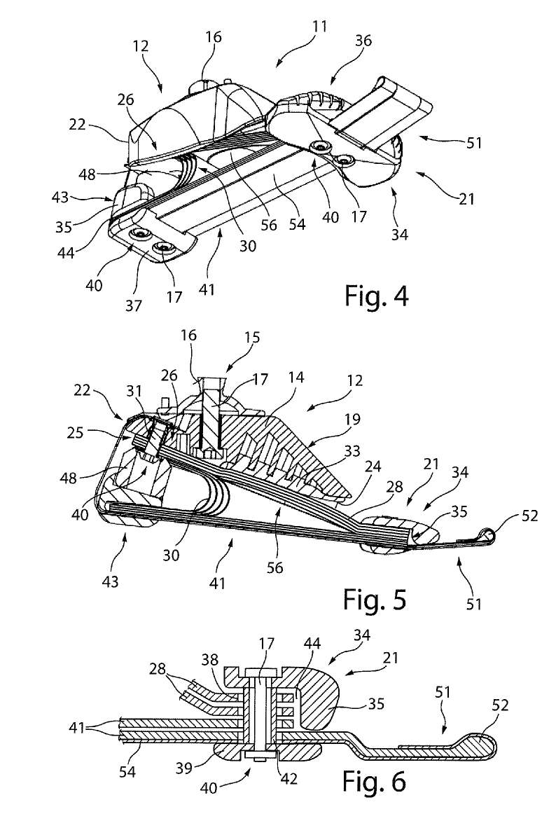

FIG. 4 shows a perspective view from the front and from below of the prosthetic foot according to FIG. 1, and

FIG. 5 shows a schematic longitudinal section of the prosthetic foot according to FIG. 1,

FIG. 6 shows a schematically enlarged sectional view of a forefoot of the prosthetic foot,

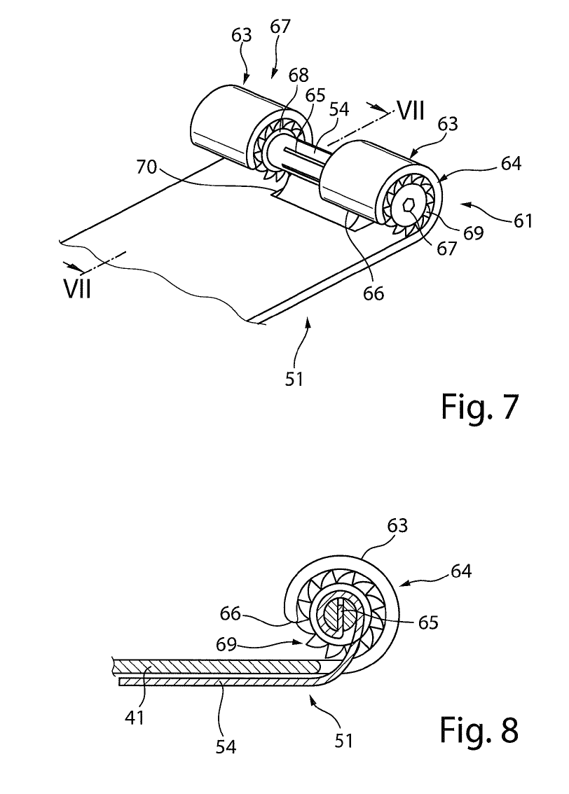

FIG. 7 shows a perspective detailed view of a pre-tensioning device for a strap for adjusting the damping in the heel region,

FIG. 8 shows a schematic sectional illustration along the line VII-VII in FIG. 7,

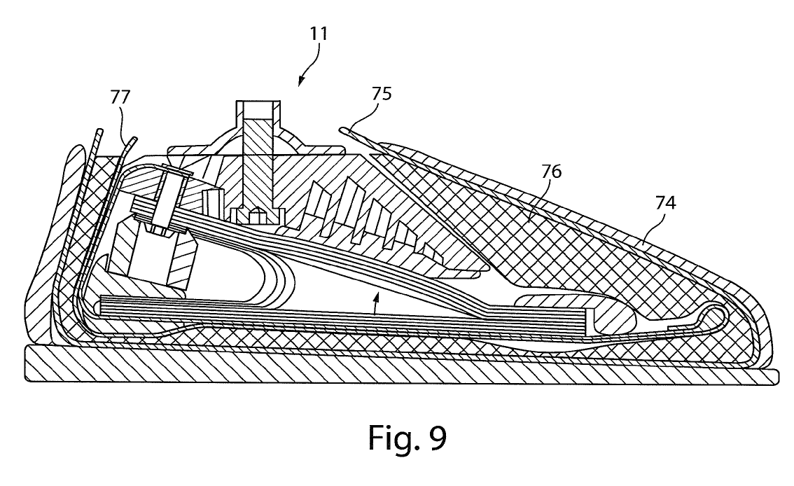

FIG. 9 shows a schematic longitudinal section of a foam-encapsulated prosthetic foot in a shoe,

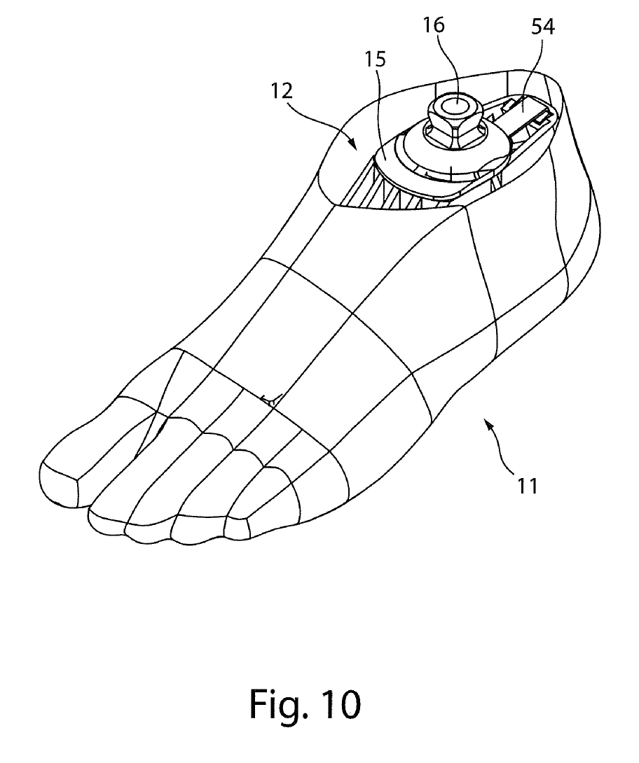

FIG. 10 shows a perspective view of a foam-encapsulated prosthetic foot with the appearance of a natural foot,

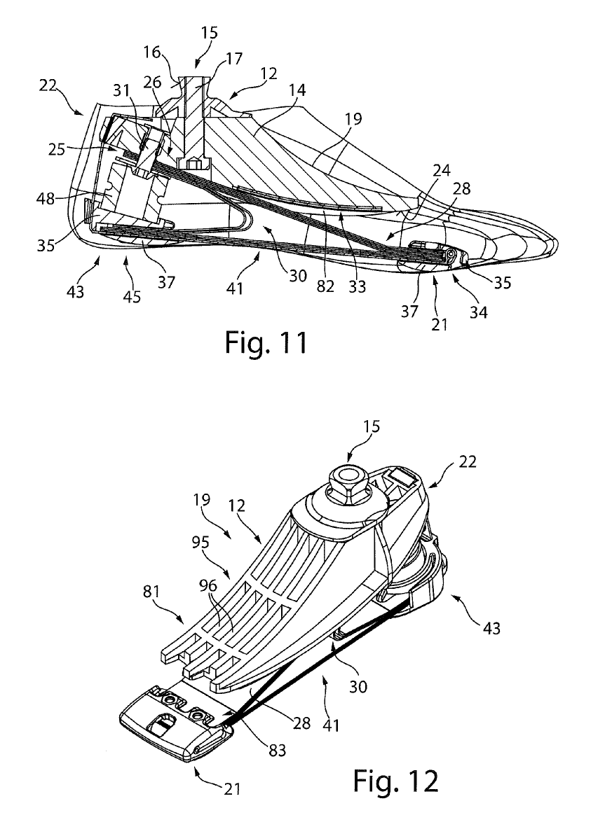

FIG. 11 shows a schematic longitudinal section of an alternative embodiment of a prosthetic foot,

FIG. 12 shows a perspective view of the alternative prosthetic foot according to FIG. 11,

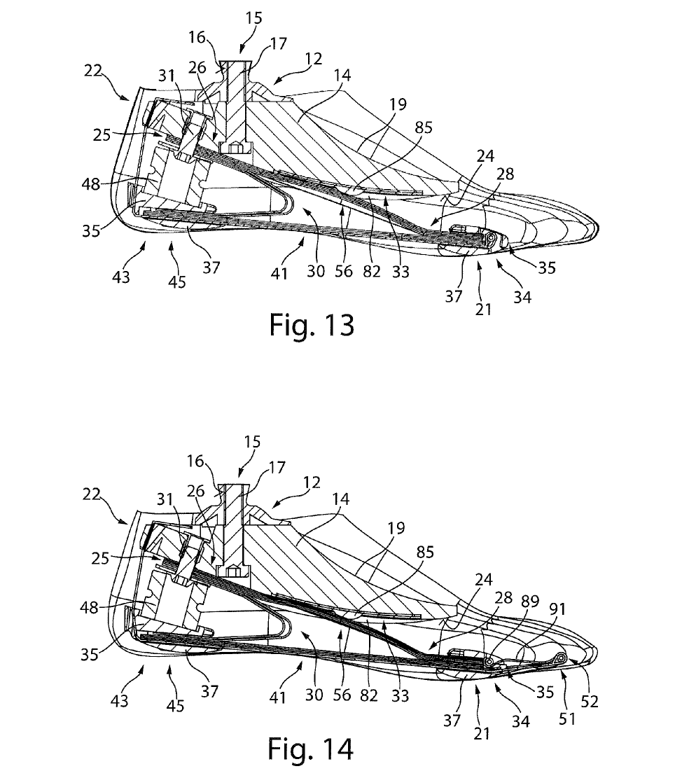

FIG. 13 shows a schematic longitudinal section of a further alternative embodiment to FIG. 11,

FIG. 14 shows a schematic longitudinal section of a further alternative embodiment to FIG. 13,

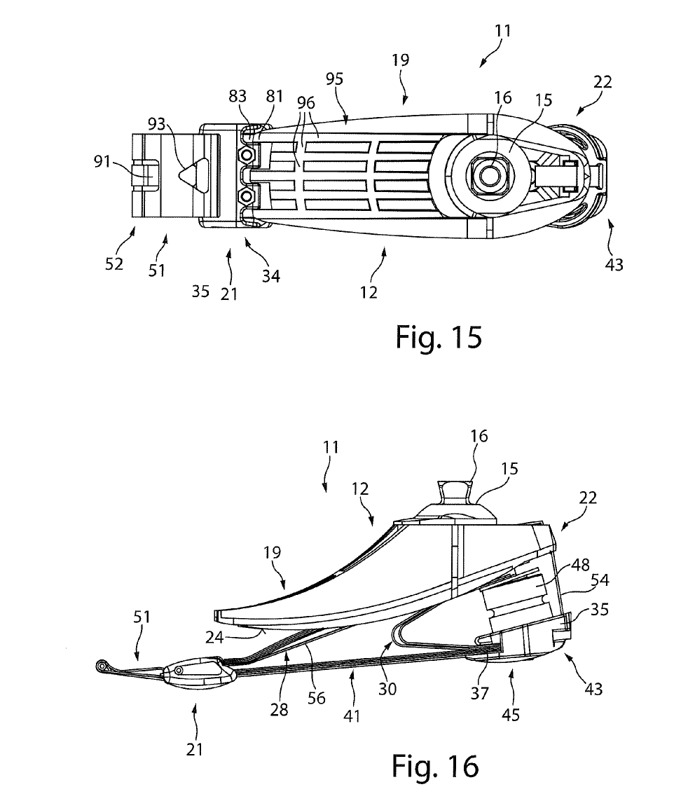

FIG. 15 shows a schematic view from above of the embodiment according to FIG. 14, and

FIG. 16 shows a schematic side view of the prosthetic foot according to FIG. 14 without foam encapsulation.

Various perspective views of a prosthetic foot 11 according to the invention are depicted in FIGS. 1 to 4. FIG. 5 shows a full cross-section in the longitudinal direction of the prosthetic foot 11. This prosthetic foot 11 comprises a housing 12, which on an upper side has a connector portion 14, in which various connectors 15 can be inserted in order to attach this prosthetic foot 11 to various below-knee prostheses, which are not depicted. For this purpose, the connection piece 16 is depicted. This connector 15 is advantageously fixed exchangeably to the housing 12 from the inside by a fastening means 17, in particular a screw, as is illustrated in the sectional depiction according to FIG. 5.

The housing 12 forms a foot upper part and comprises a foot upper side 19, which extends in the direction of a forefoot 21. This foot upper part 19 is adapted to a natural profile of an instep of the foot. Opposite the foot upper part 19, there is formed a heel region 22, which is of a short height compared to the distal end of the foot upper side 12. A bearing face 24, which has a plurality of functional portions, extends on the sole side between the heel region 22 and a distal end of the foot upper side 19. In the heel region 22, the bearing face 24 comprises a fastening portion 26, at which at least one forefoot spring element 28 and at least one heel spring element 30 are fixed by a connection means 31. The connection means 31 is preferably provided as a screw connection. Alternatively, a clip, plug, detent or clamp connection or the like can be provided. The forefoot spring element 28 preferably bears against the bearing face 24 in the region of the fastening portion 26 thereof.

The fastening portion 26 of the bearing face 24 is adjoined in the distal direction by a damping element 33, in particular a damping cushion, against which the forefoot spring element 28 pointing towards the damping element 33 comes into bearing contact at least temporarily during a walking movement, as will be described hereinafter. The damping element 33 can advantageously extend as far as the distal end of the foot upper side 19. The flexibility of the damping element 33 advantageously can be adjusted.

The forefoot 21 is freely movable in relation to the housing 12, wherein the direction of movement and the scope of movement are determined at least by the at least one forefoot spring element 28. An end portion of the at least one forefoot spring element 28 opposite the heel region 22 is received and fixed in a U-shaped or slit-like holding portion 35 of a holding device 34 on the forefoot 21. The holding device 34 by way of example can be formed in two parts, wherein said holding device comprises supporting portions 36 adjacently to the holding portion 35 and also a clamping element 39, which engages between the supporting portions 36 so as to form the holding portion 35 and receive the end portions of the at least one forefoot spring element 28 and sole spring element 41. The supporting portions 36 can comprise longitudinal ribs, which extend along a foot axis, such that a transverse stability or increase of the standing strength of the prosthetic foot 11 is attained.

The holding device 34 fixes both the at least one forefoot spring element 28 and the at least one sole spring element 41.

Opposite the forefoot 21, there is a heel 43 provided, which receives an end portion of the sole spring element 41 opposite the forefoot 21 and also an end portion of the heel spring element 30 in a holding device 45. For this purpose, the holding device 45 comprises a holding portion 35, similarly to that of the holding device 34, in which holding portion 35 the end portions of the at least one sole spring element 41 and of the at least one heel spring element 30 are received.

The end portions of the at least one forefoot spring element 28 and of the at least one sole spring element 41 arranged in the holding portion 35 of the holding device 34 preferably have slot-like cutouts 38, through which fastening means, in particular releasable fastening means 17, such as screws, are engaged so as to fix the end portions of the at least one forefoot and sole spring element 28, 41 in the receiving space 44, however receive these displaceably relative to one another. By way of example, the at least one forefoot and at least one sole spring element 28, 41 each have a slot-like cutout 38, such that the forefoot and sole spring elements 28, 41 are held by the holding portion 35 in a manner lying one on top of the other, but are displaceable relative to one another along their longitudinal axes in the holding portion 35. By way of example, a bore can also be provided in one of the two spring elements 28, 41, and a slot-like cutout can be provided in the other of the two spring elements 28, 41, such that, on the one hand, the holding device 34 is fixed to one of the two spring elements 28, 41 and the other of the two spring elements 28, 41 is displaceable relative thereto in order to attain improved roll-over properties. Alternatively to the slotlike cutouts, a U-shaped cutout can also be formed, which is open at an end side pointing into the holding portion 35. The receiving space 44 is formed by a holding portion 35 and a clamping element 39. This receiving space is open at least on one side. The clamping element 39 and the holding portion 35 are positioned relative to one another by a releasable connection 40. The releasable connection 40 is preferably formed as a screw connection, such that by way of example a threaded bolt extends through the slot-like cutouts 38 of the at least one forefoot and sole spring element 28, 41.

A sleeve 42 is fastened between the holding portion 35 and the clamping element 39 of the holding device 34, in particular is pressed or held clamped between the holding portion 35 and the clamping element 39, such that the receiving space 44 has a defined height and the forefoot and sole spring elements 28, 41 can lie loosely one on top of the other and remain displaceable relative to one another. The sleeve 42 is inserted into the slot-like cutouts 38 of the forefoot and spring sole element 28, 41. If a smaller number than the maximum number of forefoot and sole spring elements 28, 41 that can be fastened to the holding portion 35 is used, a compensation adapter can be used instead of the un-used spring element 28, 41. The space left by the absent spring element 28, 41 can thus be bridged. A compensation adapter of this type can be formed as a round disc or an insert element for fixing to the sleeve 42.

A preferred embodiment of this type is depicted schematically in an enlarged manner in FIG. 6.

The above embodiments and alternatives can apply similarly for the heel 43 and the at least one sole spring element 41 and heel spring element 30 arranged therein. However, the holding device 45 of the heel 43 preferably has a holding portion 35 and a clamping element 37, by means of which a receiving space 44 is formed, in which the end portions of the at least one sole spring element 41 and of the at least one heel spring element 30 are received and fixed firmly in relation to each other.

A damping element 48, in particular a damping rubber, is provided between the heel 43 and the heel region 22 of the housing 12 and can also be fixed to the heel region 22 by the connection means 31. This damping element is fixed opposite the heel 43 for example by clamping, or is merely supported thereon. The degree of damping can be adjusted by the selection, for example, of the Shore hardness.

At least one sole spring element 41 extends preferably through the forefoot 21 and forms a toe region 51. At the front end of the toe region 51, a rounded portion 52 is preferably provided. This toe region 51 is flexible and yielding in relation to the forefoot 21, such that this toe region 51 also constitutes a roll-over region. Alternatively, a separate toe region 51 can also be integrally formed on, or fastened to the forefoot 21.

A strap 54 extends from the toe region 51, in particular starting from an upper region of the toe region 51, over the rounded portion 52 and along an underside of the sole spring element 41 as far as the heel 43 and through this to the heel region 22 of the housing 12. This strap 54 is fixed by way of example to the toe region 51 and at the opposite end is also fixed, preferably releasably, to the fastening means 31. This strap 54 can also be fastened non-releasably in the heel region 22. This strap 54 can be fixed in such a way that a pre-tension and tensile stress acts between the two clamping points of the strap 54 in order to adjust the harmony or dynamics of the roll-over process.

In the depicted exemplary embodiment, the forefoot spring element 28, the sole spring element 41, and the heel spring element 30 are each formed as a strip-like spring element, in particular a leaf spring element, which for example has the same strip width. Alternatively, the spring elements can all differ from one another in terms of the strip width. The same is true for the material and/or the wall thickness and/or coating thereof. In order to adjust a spring characteristic curve or in order to adapt the prosthetic foot 11 to the individual wearing it or to the individual using it, one or more forefoot spring elements 28, sole spring elements 41 and/or heel spring elements 30 can be provided. By way of example, both the spring characteristic curve and the roll-over behaviour can be adjusted by the number of respective spring elements 28, 41, 30. Furthermore, the snap-action effect can be adjusted for example with regard to the number of forefoot spring elements 28 and/or the imprint formed therein, such that for example forefoot spring elements 28 with or without imprint 46 are combined with one another or forefoot spring elements 28 with different imprints are also combined with one another. By way of example, the snap-action effect during a walking movement can thus be made noticeable for the individual wearing the prosthetic foot, such that a traceable dynamic can thus be attained. Furthermore, when selecting a plurality of forefoot spring elements 28, sole spring elements 41 and/or heel spring elements 30, different strip-shaped spring elements can also be selected, which deviate from one another in terms of the material and/or the wall thickness and/or the characteristic curve.

The at least one forefoot spring element 28, the at least one sole spring element 41, and/or the at least one heel spring element 30 can be easily exchanged by the releasable connections provided at the forefoot 21 and the heel 43 and at the housing 12, so as to enable a quick and individual adaptation of the prosthetic foot to the individual wearing it. In particular, an adaptation both to the weight and to the walking habits of the individual wearing the prosthetic foot is possible. In addition, a quicker exchange is provided at the time of maintenance.

In the present exemplary embodiment, the heel spring elements 30 are U-shaped or V-shaped, wherein the region of curvature thereof points in the direction of the forefoot 21. Alternatively, they can also be V-shaped in the region of curvature. By way of example, three heel spring elements 30 are provided, which are each clamped at one end to the heel 43 and at the other end between the damping element 48 and the heel region 22. The sole spring elements 41 are advantageously formed as flat, strip-shaped spring elements, which extend in a straight line. These constitute the connection between the forefoot 21 and the heel 43. At the same time, they form a supporting face for the heel spring elements 30.

The forefoot spring elements 28 constitute a connection between the heel region 22 of the housing 12 and the forefoot 21 and can decisively determine the roll-over behaviour or the roll-over shape.

To this end, the forefoot spring elements 28 preferably have an imprint 56 in the region of the damping element 33, by means of which the spring characteristic curve of the respective forefoot spring elements 28 is determined. By providing the imprint 56, a non-linear characteristic curve is attained for the forefoot spring characteristic curve, wherein this is set in such a way that, as the individual wearing the prosthetic foot walks, a dynamic rolling of the prosthetic foot from heel to toe is made possible, wherein in particular, just before push-off via the toe region 51, a sort of catch-spring effect occurs on account of the imprint and a recovery of energy and an improved lifting of the prosthetic foot from the floor are thus obtained. The roll-over behaviour can be determined by the length of the bearing face 24 and the curvature thereof and also the selection and number of the one or more forefoot spring elements 28 and the imprint 56.

The length of the foot upper side 19 or the extent of the bearing face 24 in the direction of the forefoot 21 determines a delimitation of an acting overload and can also be adapted accordingly to the weight of the user.

The at least one forefoot spring element 28, the at least one heel spring element 30, and the at least one sole spring element 41 are directly connected to one another in the region of the fastening points thereof, i.e. in the fastening portion 26 at the heel region 22 and in the holding portions 35 of the forefoot and of the heel 43, and form a closed triangle, such that they cooperate with one another and influence one another. Here, the at least one forefoot spring element 28 and the at least one sole spring element 41 fundamentally determine the rollover behaviour, whereas the at least one heel spring element 30 fundamentally determines the cushioning as the prosthetic foot 11 touches down.

FIG. 7 shows a perspective view of a toe region 51, at which a pretensioning device 61 for the strap 54 is provided by way of example. If the pretensioning device 61 is provided at the toe region 51, the opposite end of the strap 54 can be firmly clamped to the housing 12, preferably by the releasable connection to the connection means 31. Alternatively, a pre-tensioning device can also be provided in this region, wherein the end of the strap 54 is then preferably fixed in the toe region 51 by adhesive bonding, riveting, clamping, or the like. Alternatively, a pre-tensioning device 61 can be provided at both ends.

The pre-tensioning device 61 according to FIG. 7 is depicted in a sectional depiction along the line VII-VII in FIG. 8. The toe region 51 has, at the front end, a curved end portion 63, for example curved between 180° and 360°,in particular 200° to 300°, which serves for example as a guide for a detent element 64, by means of which the strap 54 can be wound up. This detent element 64 is formed as a sort of shaft, which in the middle region has a smaller diameter of a shaft portion 68 and a slot 65, in which the free end of the strap 54 can be inserted and wedges itself as the detent element 64 rotates. The detent element 64 has a sawtooth-shaped toothing 69, which can be rotated in the clamping direction on account of the free end portion 63 and, in the opposite direction, engages an end edge 66 of an end portion 63 in a latching manner so as to thus maintain the tensile stress applied to the strap 54. By way of example, a tool holder 67, for example for an Allen key, a Torx key or a screwdriver, can be provided in the form of a slot or cross slot on an end face of the detent element 64. A recess 70 is provided between the two end portions 63, such that the strap 54 can be wound directly onto the shaft portion 68 between the sawtooth-shaped toothing 69.

By means of this pre-tensioning device 61, a pre-tension can be applied to the damping element 48 in the heel region 22 and can be permanently maintained. This pretension is adapted individually to the individual using the prosthesis and the body weight of said individual.

The above-presented and described prosthetic foot 11 can be used directly in this embodiment. However, it is preferably provided that the presented prosthetic foot 11 is encapsulated in foam and provided with a protective film, or that a foam encapsulation is provided after the prosthetic foot is surrounded by a protective or shrink film, or a rubber mixture is vulcanised onto the protective or shrink film, such that the form of a natural foot is mimicked. The foam material can serve here as an additional damping element.

FIG. 9 shows a schematic sectional view of a further alternative embodiment of the prosthetic foot 11. In this embodiment, the prosthetic foot 11 is encapsulated in foam, wherein a shoe 74 of the user is used for the shaping of the foot, such that the outer contour of the prosthetic foot 11 has a form that is accurately fitting for a shoe 74, at least for the shoe size of the individual using the prosthetic foot.

In accordance with a first embodiment, the inside of the shoe 74 is lined with a film 75, which protrudes from the shoe 74 at the area at which a foot is inserted into the shoe. The prosthetic foot 11 is then inserted into the shoe 74. The interior of the shoe 74 is then filled with foam or injected, whereby the film 75 is pressed closely against the inner walls of the shoe 74 and the prosthetic foot 11 is encased by the foam material 76 or injected material. Before the prosthetic foot 11 is encapsulated in foam, it can be directly surrounded by a protective film 77 or shrink film, such that the foam material 76 or the injection-moulding material extends externally around the prosthetic foot 11 and encases this, with the protective film 77 or shrink film being embedded. The foam material 76 thus does not infiltrate the gaps between the spring elements 28, 30, 41, such that the individual functionalities of the prosthetic foot 11 are maintained.

Once the foam material 76 or injected material has cured, the prosthetic foot 11 is removed from the shoe 74, and the film 75 is removed. The prosthetic foot 11 is thus adapted to the specific shoe size and/or a specific shoe form of the individual using the prosthetic foot.

If, when filling the shoe 74 with foam when using a prosthetic foot 11, an integral foam is used, it is sufficient as a preparatory measure that the inner walls or the interior of the shoe 74 are/is sprayed with a release agent. Here, it is not necessary to slide out the prosthetic foot with a film 75.

In FIG. 10, a perspective view of a foam-encapsulated prosthetic foot 11 is depicted, in which the foam encapsulation has the form of a natural foot. Such prosthetic feet 11 can have a rubber coating or the like on the sole or can be surrounded by an anti-slip material, such that this prosthetic foot 11 can also be formed as a barefooted prosthetic foot.

FIG. 11 shows a schematic longitudinal section of a prosthetic foot 11 according to FIG. 10. Any one of the embodiments according to FIGS. 1 to 9 and the embodiment depicted in section in FIG. 11 and presented hereinafter can be introduced within this foam-encapsulated prosthetic foot according to FIG. 10.

This embodiment according to FIG. 11 deviates from the above-described embodiment according to FIGS. 1 to 9 in that the at least one forefoot spring element 28 is formed without an imprint 56. In this case, the forefoot spring elements formed preferably in the form of leaf springs are used without an imprint.

Furthermore, this embodiment differs from the above-described embodiment in FIGS. 1 to 9 in that, instead of a damping element 33 formed as a cushion, a damping element 33 formed as a damping layer is glued onto or introduced onto the bearing face 24 of the housing 12, instead of a damping element 33 formed as a cushion. This damping element 33 formed as a damping layer can be formed from an elastomer, in particular a soft elastomer. A damping and noise reduction can be attained in turn as a result. In addition, this embodiment does not comprise a toe region 51.

FIG. 12 shows a perspective view of the prosthetic foot 11 according to FIG. 11 prior to being encapsulated by foam. The housing 12 has, at its front end pointing towards the termination 21, an end stop 81, by means of which a bending of the termination 21 in the direction of the housing 12 is delimited as the prosthetic foot rolls from heel to toe. The end stop 81 can engage here in at least one indentation 83, which is formed in the holding device 34 at the forefoot 21. As result of an end stop 81 of this type, the scope of the roll-over behaviour can be influenced. The roll-over behaviour can be influenced depending on the curvature of the bearing face 24 and/or the extent of the housing 12 in the direction of the termination 21.

In this embodiment, the bearing face 24 extends along the width of the forefoot spring element 28 and is delimited on either side by a lateral shoulder 82. These shoulders enable the at least one forefoot spring element 28 to be guided relative to the bearing face 24 as the prosthetic foot rolls from heel to toe, since the right and left shoulders 82 are each guided, respectively, along a right and left longitudinal edge of the forefoot spring element 28 during said rolling of the prosthetic foot from heel to toe.

Alternatively, at least one forefoot spring element 28 can be used with an imprint 56 in combination with a damping element 33 formed as a layer.

In this preferred embodiment, it is provided that the foot upper side 19 of the housing 12 is formed by a ribbed portion 95. This ribbed portion 95 comprises a plurality of ribs 96. Here, both longitudinal ribs and transverse ribs or a combination of longitudinal ribs and transverse ribs can be provided. The rigidity of the housing 12 can be influenced by the width, size and/or number of the ribs 96 and also the orientation thereof, that is to say the housing can have a greater or lesser resilience.

In this embodiment depicted in FIGS. 11 and 12, this foot orthosis is preferably formed without a toe region 51.

The foot orthosis depicted in FIGS. 11 and 12 is thus formed in a simplified manner on account of the omission of the imprint 56 and on account of the simplified embodiment of the damping element 33 and the omission of the toe region 51, and therefore can be produced economically.

FIG. 13 shows an alternative embodiment of the prosthetic foot 11 in relation to FIGS. 11 and 12. Compared to FIG. 11, the prosthetic foot 11 according to FIG. 13 has at least one forefoot spring element 28 with an imprint 56. In addition, a dome 85 can be provided on the bearing face 24 of the housing 12 or on a damping element 33 mounted thereon, which dome is raised in the direction of the imprint 56. A modification, in particular an increase, of the spring characteristic curve can thus be achieved selectively. In this embodiment as well, the prosthetic foot 11 is formed without the toe region 51. For the rest, reference can be made again to the above-described embodiments.

FIGS. 14 to 16 show a further alternative embodiment of the prosthetic foot in relation to FIG. 13. This prosthetic foot 11 according to FIG. 14 proceeds from the embodiment according to FIG. 13. In addition, a toe region 51 is provided in the case of this embodiment. This toe region 51 comprises an alternative embodiment compared to the embodiments depicted and described in FIGS. 5 to 8. In this embodiment, the lowermost sole spring element 41 extends through the holding device 34 in the direction of the toes. A rounded portion 52 is provided in the front region and is attained by tab portions of the sole spring element 41 that have been punched out in a U-shaped manner. A middle region between the two U-shaped limbs of the sole spring element 41 is formed as a tab element 91, which is bent back and guided back in the direction of the termination 21. This tab element acts on a stop 89 on the holding device 34. In particular, the bent-back tab element 91 engages around the stop 89. A pretension can thus be applied to the toe region 51, whereby an angular position can also be set between the orientation of the sole spring element 41 between the termination 21 and the heel 43 on the one hand and the termination 21 and the rounded portion 52 of the toe region 51 on the other hand.

The further alternative embodiments of the above-describe prosthetic feet 11 can also be provided in the case of this prosthetic foot 11.

It can also be seen from the plan view according to FIG. 15 that the sole spring element 41 has at least one recess 93 in the toe region 51. A recess 93 of this type serves to improve the resilient behaviour of the toe region 51.

It can also be seen from FIG. 15 that the end stops 81 bear against the holding device 34 in U-shaped indentations 83 and can engage therein. During a roll-over process, the termination 21 comes into bearing contact against the front region of the housing 12, wherein a further relative movement between the termination 21 and the housing 12 is delimited by the end stops 81, which come to rest in the indentations 83.

1. A prosthetic foot comprising a housing, on which a connector for a below-knee prosthesis is arrangeable, comprising a forefoot and a heel,
in which the housing and the forefoot are connected by at least one forefoot spring element,
in which the forefoot and the heel are connected by at least one sole spring element, and
in which the heel and the housing are connected by at least one heel spring element,
wherein the end portions of the at least one forefoot spring element and of the at least one sole spring element which are assigned to the forefoot are positioned in relation to one another, by means of at least one holding device, in such a way that the at least one forefoot spring element and the at least one sole spring element are guided displaceably relative to one another along their longitudinal axis during a roll-over movement.

2. The prosthetic foot according to claim 1, wherein the end portions of the at least one sole spring element and of the at least one heel spring element which are assigned to the heel are positioned firmly in relation to one another by means of at least one holding device, in the holding device.

3. The prosthetic foot according to claim 1, wherein the holding device of the forefoot and the holding device of the heel have at least one slit-like or U-shaped holding portion for receiving the respective end portions of the at least one forefoot spring element and sole spring element, and of the at least one sole spring element and heel spring element.

4. The prosthetic foot according to claim 1, wherein the end portions of the at least one forefoot spring element and of the at least one sole spring element which are assigned to the forefoot have a slot-like cutout or a U-shaped cutout, through which a fastening means of the holding device for positioning the forefoot spring element and sole spring element in the holding device extends.

5. The prosthetic foot according to claim 4, wherein the holding device of the forefoot comprises at least one holding portion and a clamping element, which form a receiving space for the end portions of the at least one forefoot spring element and the at least one sole spring element, and the holding portion and the clamping element are arrangeable at a predetermined distance from one another, and a sleeve is held clamped therebetween.

6. The prosthetic foot according to claim 1, wherein the end portions of the at least one forefoot spring element and of the sole spring element are fastened, in the forefoot, the end portions of the at least one sole spring element and of the heel spring element are fastened, in the heel, and the end portions of the at least one heel spring element and of the at least one forefoot spring element are fastened, at the housing.

7. The prosthetic foot according to claim 1, wherein the at least one forefoot spring element, the at least one sole spring element, and the at least one heel spring element form a fundamentally isosceles triangle which is formed by the at least one forefoot spring element and the at least one sole spring element, and the at least one heel spring element has a V-shaped extent, wherein the region of curvature of the V-shaped region points in the direction of the forefoot.

8. (canceled)

9. The prosthetic foot according to claim 1, wherein the at least one forefoot spring element, the at least one sole spring element, and/or the at least one heel spring element are/is formed as a strip-shaped spring element or forged leaf spring element.

10. (canceled)

11. The prosthetic foot according to claim 1, wherein the housing is formed as a foot upper part which has a connector portion, from which a foot upper side extends in a wedge-like manner in the direction of the forefoot and which comprises a bearing face arranged on the sole side, which bearing face extends from the distal end of the foot upper side to the heel region, which has a curvature, in a distal region, for forming a roll-over radius for the at least one forefoot spring element and runs in a rising manner to the heel region.

12. The prosthetic foot according to claim 11, wherein the housing has an end stop distally on the bearing face, which end stop delimits a curvature or bending of the forefoot spring element during a roll-over movement.

13. The prosthetic foot according to claim 1, wherein a dome directed towards the forefoot spring element is provided on the bearing face of the housing.

14-18. (canceled)

19. The prosthetic foot according to claim 11, wherein the at least one forefoot spring element extends freely to the forefoot starting from a fastening portion on the bearing face in the heel region, and the at least one forefoot spring element comes completely into abutment against the bearing face during a roll-over movement of the prosthetic foot.

20. The prosthetic foot according to claim 19, wherein a damping element forming the bearing face is provided between the fastening portion and a distal end of the bearing face.

21. The prosthetic foot according to claim 21, wherein the at least one forefoot spring element has the an imprint, which is directed in the direction of the damping element, at least in the region of the damping element forming the bearing face.

22. The prosthetic foot according to claim 1, wherein at least one sole spring element extends beyond the forefoot and forms a toe region and a strap acts on the front end of the toe region and extends along an underside of the sole spring element as far as the heel and through this or around this as far as the heel region of the housing, and is releasably fixed at the heel region.

23-24. (canceled)

25. The prosthetic foot according to claim 1, wherein the at least one forefoot spring element, sole spring element, and/or heel spring element are/is formed from high-grade steel, an aluminium alloy, a composite material, a steel alloy, or a soft metal.

26. The prosthetic foot according to claim 1, wherein the housing and/or the forefoot and/or the heel are/is produced from a plastic from a composite material or a fibre-reinforced plastic, or from a light metal.

27. The prosthetic foot according to claim 1, wherein the forefoot, transversely to the sole longitudinal direction, is wider than the housing and/or the heel and the forefoot has resiliently flexible supporting portions on one or both sides outside the holding portion for the end portions of the at least one forefoot spring element and sole element.

28-29. (canceled)

30. The prosthetic foot according to claim 1, wherein the housing has, at least on the foot upper side, a ribbed portion comprising a plurality of ribs, wherein the rigidity of the housing is adjusteable by the number and/or size and/or extent of the ribs.

31. A method for producing a prosthetic foot wherein:
at least one forefoot spring element and heel spring element are connected to a housing,
at least one heel spring element and sole spring element are connected to one another, and
at least one forefoot spring element and at least one sole spring element are connected to one another,
wherein the at least one heel sprig element and the at least one forefoot spring element, the at least one forefoot spring element and the at least one sole spring element, and the at least one sole spring element and the at least one heel spring element are connected to one another by means of a releasable connection and the at least one forefoot spring element, the at least one sole spring element, and the at least one heel spring element are encased by a protective film and then the assembly is provided at least approximately with the form of a natural foot by being covered in foam or vulcanised.

32-34. (canceled)

35. The prosthetic foot according to claim 25, wherein the at least one forefoot spring element, sole spring element, and/or heel spring element has a coating which is formed as a permanent coating or as a removable coating.
